(19) United States
(12) Patent Application Publication
(10) Pub. No.: US 2018/0010158 A1
(43) Pub. Date: Jan. 11, 2018

(54) A PROCESS FOR THE PREPARATION OF TOFACITINIB CITRATE

(71) Applicant: SUN PHARMACEUTICAL INDUSTRIES LIMITED, Mumbai, Maharashtra (IN)

(72) Inventors: Somashekar Rudrappa BHANDYA, Shivamogga (IN); Mukesh Kumar MADHRA, Karnal (IN)

(21) Appl. No.: 15/552,392

(22) PCT Filed: Feb. 22, 2016
(86) PCT No.: PCT/IB16/50946

§ 371 Date: Aug. 21, 2017

(30) Foreign Application Priority Data
Feb. 20, 2015 (IN) 490/DEL/2015

(51) Int. Cl. C12P 17/18; C07D 487/04

(52) U.S. Cl. CPC C12P 17/182; C12Y 304/21062; C07D 487/04; C12Y 301/01003

(57) ABSTRACT

The present invention provides a process for the preparation of tofacitinib citrate of Formula I. Specifically, the present invention provides an enzymatic route for the preparation of tofacitinib of Formula II, which is converted to tofacitinib citrate of Formula I.

### FIELD OF THE INVENTION

The present invention provides a process for the preparation of tofacitinib citrate of Formula I. Specifically, the present invention provides an enzymatic route for the preparation of tofacitinib of Formula II, which is converted to tofacitinib citrate of Formula I.

### BACKGROUND OF THE INVENTION

Tofacitinib citrate chemically is (3R,4R)-4-methyl-3-(methyl-7H-pyrrolo [2,3-d]pyrimidin-4-ylamino)-β-oxo-1-piperidinepropanenitrile, 2-hydroxy-1,2,3-propanetricarboxylate (1:1), represented by Formula I.

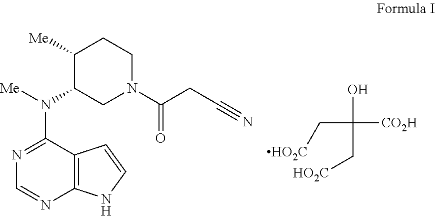

Tofacitinib citrate is an inhibitor of Janus kinases (JAK).

U.S. Pat. No. RE41,783; Chinese Patent Nos. CN 102875555, CN 104059016, CN 103819474; and PCT Publication No. WO 2014/083150 provide chemical routes for the preparation of tofacitinib citrate. In these processes, the coupling of secondary amines with ethyl cyanoacetate or cyanoacetic acid or cyanoacetyl chloride is carried out in the presence of substrate activating agents such as 1,8-diazabicyclo[5.4.0]undec-7-ene or 1-ethyl-3-(3-dimethylaminopropyl)carbodiimide hydrochloride/hydroxybenzotriazole, which are genotoxic in nature. Therefore, there is a need in the art to develop a process which avoids the use of these genotoxic chemicals.

### SUMMARY OF THE INVENTION

The present invention provides a process for the preparation of tofacitinib citrate of Formula I. Specifically, the present invention provides an enzymatic route for the preparation of tofacitinib of Formula II, which is converted to tofacitinib citrate of Formula I. The process of the present invention is simple and environmentally friendly as it avoids the use of genotoxic chemicals, which are required for substrate activation in the chemical route. The process of the present invention is commercially viable as it makes use of inexpensive enzymes. Tofacitinib citrate obtained by following the process of the present invention has high purity.

### DETAILED DESCRIPTION OF THE INVENTION

The term “about,” as used herein, refers to any value which lies within the range defined by a number up to ±10% of the value.

The term “lower alkyl,” as used herein, refers to both straight chain and branched chain alkyl groups having 1 to 6 carbon atoms. Examples of lower alkyls include methyl, ethyl, propyl, iso-propyl, n-butyl, sec-butyl, iso-butyl, tent-butyl, n-pentyl, iso-pentyl, n-hexyl, and iso-hexyl.

The term “room temperature,” as used herein, refers to a temperature in the range of 25° C. to 35° C.

A first aspect of the present invention provides a process for the preparation of tofacitinib of Formula II,

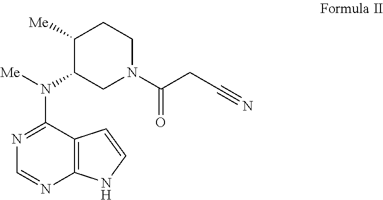 [SMILES: C[C@@H]1CCN(C(=O)CC#N)C[C@@H]1N(C)c1ncnc2[nH]ccc12]

comprising reacting a compound of Formula III

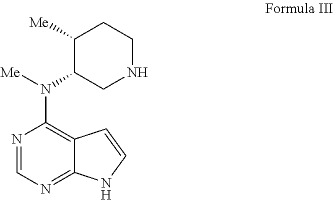 [SMILES: C[C@@H]1CCNC[C@@H]1N(C)c1ncnc2[nH]ccc12]

with a compound of Formula IV,

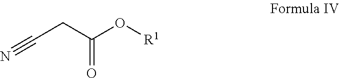

wherein R¹ is selected from hydrogen or lower alkyl,
in the presence of an enzyme.

A second aspect of the present invention provides a process for the preparation of tofacitinib citrate of Formula I,

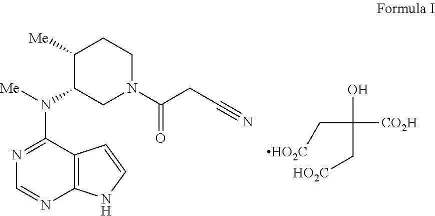

comprising:

a) reacting a compound of Formula III

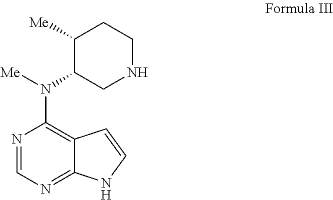 [SMILES: C[C@@H]1CCNC[C@@H]1N(C)c1ncnc2[nH]ccc12]

with a compound of Formula IV,

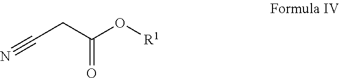

wherein R¹ is selected from hydrogen or lower alkyl,
in the presence of an enzyme to obtain tofacitinib of Formula II; and

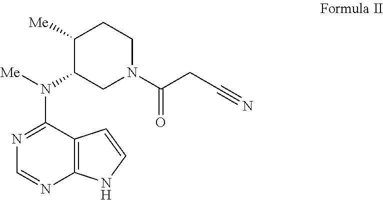 [SMILES: C[C@@H]1CCN(C(=O)CC#N)C[C@@H]1N(C)c1ncnc2[nH]ccc12]

b) converting tofacitinib of Formula II into tofacitinib citrate of Formula I.

The compound of Formula III can be prepared by following the methods provided in the art, for example, U.S. Pat. No. 7,301,023 or PCT Publication No. WO 2007/012953.

The reaction of the compound of Formula III with the compound of Formula IV to obtain tofacitinib of Formula II is carried out in the presence of an enzyme in a solvent in the optional presence of molecular sieves.

The enzyme is selected from the group consisting of lipases and proteases. Examples of lipases include Lipozyme® RM IM, Novozym® 435, Savinase® 12T, Lipozyme® TL IM, Lipase PS “Amano®” SD, Lipase AS “Amano®,” Acylase “Amano®,” SPRIN Anti CAL, immobilized Candida antarctica lipase B adsorbed on a highly hydrophobic polymer, lipase from Rhizopus arrhizus, lipase from Candida cylindracea, lipase from Candida antarctica, Addzyme TL 165G, Addzyme RD 165G, Addzyme CALB 165G, FermaseCALB™ 10000, and adsorbed CALB. Examples of proteases include Protease S “Amano®”, Protease N “Amano®”, and Subtilisin A.

Preferably, the enzymes are Novozym® 435, Savinase® 12T, and Addzyme RD 165G.

The solvent is selected from the group consisting of hydrocarbons, halogenated hydrocarbons, ethers, ketones, esters, alcohols, amides, dimethyl sulfoxide, and mixtures thereof.

Examples of hydrocarbons include toluene, hexane, heptane, cyclohexane, cyclopentane, cycoheptane, benzene, xylene, and mixtures thereof.

Examples of halogenated hydrocarbons include dichloromethane, chloroform, carbon tetrachloride, chloroethane, and mixtures thereof.

Examples of ethers include dioxane, tetrahydrofuran, methyl tetrahydrofuran, diisopropyl ether, diethyl ether, diglyme, di-tent-butyl ether, dimethoxyethane, methyl tert-butyl ether, tetrahydropyran, and mixtures thereof.

Examples of ketones include acetone, methyl tent-butyl ketone, methyl isobutyl ketone, butanone, cyclopentanone, methyl isopropyl ketone, ethyl isopropyl ketone, 2-hexanone, and mixtures thereof.

Examples of esters include tent-butyl acetate, ethyl acetate, butyl acetate, isopropyl acetate, isoamyl acetate, isobutyl acetate, methyl acetate, and mixtures thereof.

Examples of alcohols include tent-butanol, benzyl alcohol, n-butanol, methanol, ethanol, propanol, isopropanol, isobutanol, diethylene glycol, ethylene glycol, furfuryl alcohol, glycerol, 2-pentanol, and mixtures thereof.

Examples of amides include dimethylformamide, dimethylacetamide, formamide, and mixtures thereof.

Preferably, the solvents are toluene, tetrahydrofuran, hexane, dimethyl sulfoxide, and mixtures thereof.

The reaction of the compound of Formula III with the compound of Formula IV is carried out for about 25 hours to about 80 hours, for example, for about 26 hours to about 75 hours.

The reaction of the compound of Formula III with the compound of Formula IV is carried out at a temperature of about 50° C. to about 80° C., for example, of about 60° C. to about 75° C.

Tofacitinib of Formula II may optionally be isolated by filtration, decantation, extraction, distillation, evaporation, chromatography, precipitation, concentration, crystallization, centrifugation, or recrystallization. Tofacitinib of Formula II may be dried using conventional techniques, for example, drying, drying under vacuum, spray drying, air drying, or agitated thin film drying.

Tofacitinib of Formula II can be converted to tofacitinib citrate of Formula I by any of the methods described in the art, for example, as in U.S. Pat. No. RE41,783; Chinese Patent Nos. CN 102875555, CN 104059016, CN103819474; and PCT Publication No. WO 2014/102826, or by using the methods described herein.

While the present invention has been described in terms of its specific aspects and embodiments, certain modifications and equivalents will be apparent to those skilled in the art and are intended to be included within the scope of the present invention.

The following examples are for illustrative purposes only and should not be construed as limiting the scope of the invention in any way.

### Methods

HPLC purity was determined using a Waters Alliance® 2695 HPLC instrument.

### EXAMPLES

### Example 1

### Preparation of Tofacitinib (Formula II)

N-Methyl-N-[(3R,4R)-4-methylpiperidin-3-yl]-7H-pyrrolo[2,3 -d]pyrimidin-4-amine (Formula III, 2 g), ethyl cyanoacetate (Formula IV, wherein R¹ is ethyl, 2.2 g), activated molecular sieves 4 Å (0.52 g), and Novozym® 435 (0.2 g) were added to toluene (50 mL) at room temperature under inert atmosphere. The reaction mixture was stirred at 65° C. to 70° C. under inert atmosphere for 48 hours, and then cooled to room temperature. Dichloromethane (30 mL) and methanol (10 mL) were added to the reaction mixture, then the reaction mixture was stirred at room temperature for 15 minutes, and then filtered off through a Hyflo®. The filtrate obtained was distilled off under vacuum at 45° C. to obtain an oily residue (3.04 g) of the title compound, which was used as such for the next step.

### Example 2

### Preparation of Tofacitinib Citrate (Formula I)

The oily residue (3.04 g, as obtained in Example 1) was dissolved in methanol (15 mL), and then a citric acid solution (1.79 g citric acid monohydrate in 4 mL deionized water) was slowly added to the mixture. The reaction mixture was stirred at room temperature for 5 hours, then filtered off, and then dried to obtain a crude material (1.35 g). The crude material was suspended in a mixture of methanol (32 mL) and deionized water (10 mL), and then the mixture was heated to reflux for 10 minutes. The resulting mixture was cooled to room temperature, then filtered, then washed with a methanol (11 mL) and water (3 mL) mixture, and then dried to obtain the title compound.

### Yield: 0.8 g

HPLC purity: 99.04%

### Example 3

### Preparation of Tofacitinib (Formula II)

N-Methyl-N-[(3R,4R)-4-methylpiperidin-3-yl]-7H-pyrrolo[2,3 -d]pyrimidin-4-amine (Formula III, 5 g), ethyl cyanoacetate (Formula IV, wherein R¹ is ethyl, 10 g), activated molecular sieves 4 Å (1 g), and Addzyme RD 165G (0.5 g) were added to tetrahydrofuran (50 mL) at room temperature under inert atmosphere. The reaction mixture was stirred at 65° C. to 70° C. for 28 hours, and then cooled to room temperature. Dichloromethane (75 mL) was added to the reaction mixture, and then the reaction mixture was filtered off The filtrate obtained was distilled off under reduced pressure to give an oily residue (13.2 g) of the title compound, which was used as such for the next step.

### Example 4

### Preparation of Tofacitinib Citrate (Formula I)

The oily residue (13.2 g, as obtained in Example 3) was dissolved in methanol (37.5 mL), and then a citric acid solution (4.5 g citric acid monohydrate in 10 mL deionized water) was added to the mixture. The reaction mixture was stirred at room temperature for 4 hours, then filtered off, then washed with methanol (10 mL), and then dried to obtain a crude material (6.5 g). The crude material thus obtained was suspended in a mixture of methanol (168 mL) and deionized water (56 mL) at room temperature, then the mixture was heated to reflux, and then charcoalized. The resulting mixture was filtered off through a Hyflo® and again washed with a methanol:water mixture (7.5:2.5 v/v, 12.8 mL). The filtrate was cooled to room temperature. The mixture was further cooled to 15° C. to 20° C., and then stirred for 60 minutes at 15° C. to 20° C. The solid precipitate obtained was filtered, then washed with a methanol (9.6 mL) and water (3.2 mL) mixture, and then dried to obtain the title compound.

### Yield: 4.5 g

HPLC purity: 98.71%.

### Example 5

### Preparation of Tofacitinib (Formula II)

N-Methyl-N-[(3R,4R)-4-methylpiperidin-3-yl]-7H-pyrrolo[2,3 -d]pyrimidin-4-amine (Formula III, 5 g), ethyl cyanoacetate (Formula IV, wherein R¹ is ethyl, 10 g), activated molecular sieves 4 Å (1 g), and Savinase® 12T (0.5 g) were added to tetrahydrofuran (50 mL) at room temperature under nitrogen atmosphere. The reaction mixture was stirred at 65° C. to 70° C. for 28 hours, and then cooled to room temperature. Dichloromethane (75 mL) was added to the reaction mixture, and then the reaction mixture was filtered off The filtrate obtained was distilled off under reduced pressure to give an oily residue (13.2 g) of the title compound, which was used as such for the next step.

### Example 6

### Preparation of Tofacitinib Citrate (Formula I)

The oily residue (13.2 g, as obtained in Example 5) was dissolved in methanol (37.5 mL), and then a citric acid solution (4.5 g citric acid monohydrate in 10 mL deionized water) was added to the mixture. The reaction mixture was stirred at room temperature for 4 hours, then filtered, then washed with methanol (10 mL), and then dried to obtain a crude material (5.1 g). The crude material thus obtained was suspended in a mixture of methanol (131 mL) and deionized water (43.7 mL) at room temperature, and then the mixture was heated to reflux, and then charcoalized. The reaction mixture was filtered off through a Hyflo®, and then washed with a methanol:water mixture (7.5:2.5 v/v, 10 mL). The filtrate was cooled to room temperature. The mixture was further cooled to 15° C. to 20° C., and then stirred for 60 minutes at 15° C. to 20° C. The solid precipitate obtained was filtered, then washed a methanol:water mixture (7.5:2.5 v/v, 10 mL), and then dried to obtain the title compound.

### Yield: 3.5 g

HPLC purity: 98.89%.

### Example 7

### Preparation of Tofacitinib (Formula II)

N-Methyl-N-[(3R,4R)-4-methylpiperidin-3-yl]-7H-pyrrolo [2,3 -d]pyrimidin-4-amine (Formula III, 5 g), ethyl cyanoacetate (Formula IV, wherein R¹ is ethyl, 10 g), activated molecular sieves 4 Å (1 g), and Novozym® 435 (0.5 g) were added to a dimethyl sulfoxide:hexane mixture (1:10 v/v, 55 mL) at room temperature under inert atmosphere. The reaction mixture was stirred at 65° C. to 70° C. for 28 hours, followed by cooling to room temperature. Dichloromethane (75 mL) was added to the reaction mixture, and then the reaction mixture was filtered off. The filtrate was distilled off under reduced vacuum to give an oily residue (16.5 g) of the title compound, which was used as such for the next step.

### Example 8

### Preparation of Tofacitinib Citrate (Formula I)

The oily residue (16.5 g, as obtained in Example 7) was dissolved in methanol (37.5 mL), and then a citric acid solution (4.5 g citric acid monohydrate in 10 mL deionized water) was added to the mixture. The reaction mixture was stirred at room temperature for 4 hours, then filtered, then washed with methanol (10 ml), and then dried to obtain a crude material (6.5 g). The crude material was suspended in mixture of methanol (91.8 mL) and deionized water (30.6 mL) at room temperature, then heated to reflux, and then charcoalized. The mixture was then filtered off through a Hyflo® and washed with a methanol:water mixture (7.5:2.5 v/v, 10 mL). The filtrate was cooled to room temperature, then further cooled to 15° C. to 20° C., and then stirred for 60 minutes at 15° C. to 20° C. The solid precipitate obtained was filtered, then washed a methanol:water mixture (7.5:2.5 v/v, 10 mL), and then dried to obtain the title compound.

### Yield: 2.2 g

HPLC purity: 97.87%.

### Example 9

### Preparation of Tofacitinib (Formula II)

N-Methyl-N-[(3R,4R)-4-methylpiperidin-3-yl]-7H-pyrrolo[2,3 -d]pyrimidin-4-amine (Formula III, 5 g), ethyl cyanoacetate (Formula IV, wherein R¹ is ethyl, 10 g), activated molecular sieves 4 Å (1 g), and Savinase® 12T (0.5 g) were added to toluene (50 mL) at room temperature under inert atmosphere. The reaction mixture was stirred at 65° C. to 70° C. for 28 hours, followed by cooling to room temperature. Dichloromethane (75 mL) was added to the reaction mixture, and then the reaction mixture was filtered off. The filtrate obtained was distilled off under reduced vacuum to obtain an oily residue (12.8 g) of the title compound, which was used as such for the next step.

### Example 10

### Preparation of Tofacitinib Citrate (Formula I)

The oily residue (12.8 g, as obtained in Example 9) was dissolved in methanol (37.5 mL), and then a citric acid solution (4.5 g citric acid monohydrate in 10 mL deionized water) was added to the mixture. The mixture was stirred at room temperature for 4 hours, then filtered, then washed with methanol (10 mL), and then dried to obtain a crude material (4.2 g). The crude material obtained was suspended in mixture of methanol (105 mL) and deionized water (35 mL) at room temperature, then heated to reflux, and then charcoalized. The mixture was then filtered through a Hyflo® and washed with a methanol:water mixture (7.5:2.5 v/v, 10 mL). The filtrate was cooled to room temperature, then further cooled to 15° C. to 20° C., and then stirred for 60 minutes at 15° C. to 20° C. The solid precipitate was filtered, then washed with a methanol:water mixture (7.5:2.5 v/v, 10 mL), and then dried to obtain the title compound.

### Yield: 2.1 g

HPLC purity: 97.28%.

### Example 11

### Preparation of Tofacitinib Citrate (Formula I)

N-Methyl-N-[(3R,4R)-4-methylpiperidin-3-yl]-7H-pyrrolo[2,3 -d]pyrimidin-4-amine (Formula III, 10 g), ethyl cyanoacetate (Formula IV, wherein R¹ is ethyl, 7.0 g), and Addzyme RD 165G (0.47 g) were added to tetrahydrofuran (100 mL) at room temperature under inert atmosphere. The reaction mixture was added to a mixture of activated molecular sieve 4 Å powder and a Hyflo® (1:1 w/w, 37 g), and then stirred at 65° C. to 70° C. for 68 hours, followed by cooling to room temperature. A citric acid solution (17 g in 60 mL deionized water) was added to the reaction mixture, and then the mixture was stirred at room temperature for 1 hour. The reaction mixture was cooled to 0° C. to 5° C., stirred for 3 hours, then filtered, and then washed with a tetrahydrofuran:water mixture (1:1 v/v, 40 mL) to obtain a crude material (17.7 g). The crude compound (17 g) was suspended in a mixture of methanol (297 mL) and deionized water (297 mL), then heated, then charcoalized, then filtered, and then cooled to room temperature. The mixture was further cooled to 0° C. to 5° C., and then stirred at this temperature for 3 hours. The solid was filtered, and then washed with a methanol:water mixture (1:1 v/v, 40 mL) to obtain the title compound (14.3 g).

Tofacitinib citrate as obtained above (13 g) was suspended in a mixture of methanol (227 mL) and deionized water (227 mL) at room temperature. The mixture was further heated to reflux, and then stirred at this temperature for 1 hour. The obtained mixture was then cooled to 0° C. to 5° C., and then stirred for 3 hours at 0° C. to 5° C. The precipitated solid was filtered, then washed with methanol:water mixture (1;1 v/v, 40 mL), and then dried.

### Yield: 11.6 g

HPLC purity: 99.44%.

1. A process for the preparation of tofacitinib of Formula II, 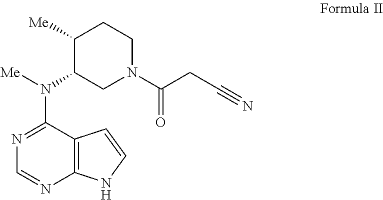 [SMILES: C[C@@H]1CCN(C(=O)CC#N)C[C@@H]1N(C)c1ncnc2[nH]ccc12]
comprising reacting a compound of Formula III 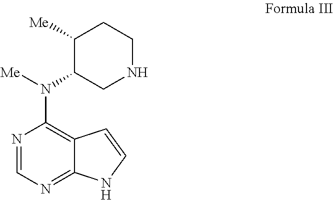 [SMILES: C[C@@H]1CCNC[C@@H]1N(C)c1ncnc2[nH]ccc12]
with a compound of Formula IV, 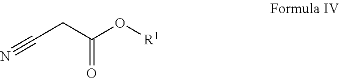
wherein R¹ is selected from hydrogen or lower alkyl,
in the presence of an enzyme.

2. The process according to claim 1, wherein the tofacitinib of Formula II is further converted to tofacitinib citrate of Formula I

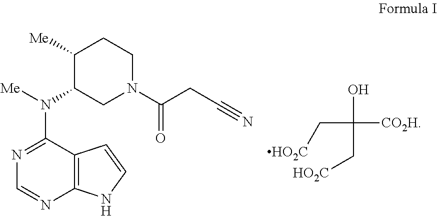

3. The process according to claim 1, wherein the enzyme is selected from the group consisting of lipases and proteases.

4. The process according to claim 3, wherein the lipases are selected from the group consisting of Lipozyme® RM IM, Novozym® 435, Savinase® 12T, Lipozyme® TL IM, Lipase PS “Amano®” SD, Lipase AS “Amano®,” Acylase “Amano®,” SPRIN Anti CAL, immobilized Candida antarctica lipase B adsorbed on a highly hydrophobic polymer, lipase from Rhizopus arrhizus, lipase from Candida cylindracea, lipase from Candida antarctica, Addzyme TL 165G, Addzyme RD 165G, Addzyme CALB 165G, FermaseCALB™ 10000, and adsorbed CALB.

5. The process according to claim 3, wherein the proteases are selected from the group consisting of Protease S “Amano®,” Protease N “Amano®,” and Subtilisin A.

6. The process according to claim 1, wherein the reaction of the compound of Formula III with the compound of Formula IV is carried out in a solvent.

7. The process according to claim 6, wherein the solvent is selected from the group comprising of hydrocarbons, halogenated hydrocarbons, ethers, ketones, esters, alcohols, amides, dimethyl sulfoxide, and mixtures thereof.

8. The process according to claim 7, wherein the hydrocarbons are selected from the group consisting of toluene, hexane, heptane, cyclohexane, cyclopentane, cycoheptane, benzene, xylene, and mixtures thereof.

9. The process according to claim 7, wherein the halogenated hydrocarbons are selected from the group consisting of dichloromethane, chloroform, carbon tetrachloride, chloroethane, and mixtures thereof.

10. The process according to claim 7, wherein the ethers are selected from the group consisting of dioxane, tetrahydrofuran, methyl tetrahydrofuran, diisopropyl ether, diethyl ether, diglyme, di-tent-butyl ether, dimethoxyethane, methyl tent-butyl ether, tetrahydropyran, and mixtures thereof.

11. The process according to claim 7, wherein the ketones are selected from the group consisting of acetone, methyl tent-butyl ketone, methyl isobutyl ketone, butanone, cyclopentanone, methyl isopropyl ketone, ethyl isopropyl ketone, 2-hexanone, and mixtures thereof.

12. The process according to claim 7, wherein the esters are selected from the group consisting of tent-butyl acetate, ethyl acetate, butyl acetate, isopropyl acetate, isoamyl acetate, isobutyl acetate, methyl acetate, and mixtures thereof.

13. The process according to claim 7, wherein the alcohols are selected from the group consisting of tent-butanol, benzyl alcohol, n-butanol, methanol, ethanol, propanol, isopropanol, isobutanol, diethylene glycol, ethylene glycol, furfuryl alcohol, glycerol, 2-pentanol, and mixtures thereof.

14. The process according to claim 7, wherein the amides are selected from the group consisting of dimethylformamide, dimethylacetamide, formamide, and mixtures thereof.

15. The process according to claim 1, wherein the reaction of the compound of Formula III with the compound of Formula IV is carried out in the presence of molecular sieves.
